(19) United States
(12) Patent Application Publication
(10) Pub. No.: US 2006/0195043 A1
(43) Pub. Date: Aug. 31, 2006

(54) Methods and apparatus for measuring pressures in bodily fluids

(75) Inventors: Sherman L. Rutherford, Portola Valley, CA (US); Philip C. Rutherford, Park City, UT (US); Ronald M. Paitich, Auburn, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: 101 Associates, Portola Valley, CA (US)

(21) Appl. No.: 11/069,528

(22) Filed: Feb. 28, 2005

(51) Int. Cl. A61B 5/00

(52) U.S. Cl. 600/561

(57) ABSTRACT

Various embodiments of the invention, therefore, provide enhanced devices for the measurement of a pressure of a bodily fluid, as well as methods of using and/or manufacturing such devices. In one set of embodiments, for example, a device for measuring a fluid may comprise an inlet port, which may be configured to be in fluid communication with a bodily fluid. Merely by way of example, in a particular set of embodiments, the inlet port may be in fluid communication with (and/or may comprise and/or be incorporated within) a needle designed to be inserted (e.g., subdermally) into a subject (such as a human and/or animal patient).

### BACKGROUND OF THE INVENTION

The present invention relates generally to the measurement of pressures in fluids. More specifically, embodiments of the invention relate to the measurement of pressures in bodily fluids, including without limitation human bodily fluids.

In the field of medical diagnosis and treatment, it is often useful to ascertain the pressure of a particular fluid. Merely by way of example, the pressure of a patient's cerebrospinal fluid often can be used by a physician, pathologist, and/or the like to identify certain conditions in the patient.

In the past, a conventional capillary manometer often has been used to perform such measurements. For example, FIG. 1 illustrates a conventional device 100 used to measure the pressure of a patient's cerebrospinal fluid. The device 100 comprises a needle 105 inserted into the subarachnoid space, most commonly accessed in the lumbar region of a patient 110, and/or in other locations where cerebrospinal fluid circulates. The needle is in fluid communication with a valve 115, which provides fluid communication with a manometer 120. When the valve 115 is opened, cerebrospinal fluid enters the manometer 120 via the needle 105, rising to a level corresponding to the pressure of the cerebrospinal fluid. Typically, the manometer 120, will have gradations 125 calibrated to indicate the absolute pressure of the fluid.

Often, however, to produce readable results, the manometer 120 will have to be relatively large. Merely by way of example, a typical manometer used for this purpose is approximately 55 cm in height. The height of the manometer 120 often makes such a device 100 difficult to use, in that a physician (or any other user) must hold the manometer 120 steady enough to read while at the same time stabilizing the needle 105 to prevent injury and/or discomfort for the patient 110, all while attempting to read the manometer 120 to determine the pressure of the patient's spinal fluid. This can result in an inconvenience for the physician, potential discomfort or injury for the patient, and a substantial risk that the manometer 120 might be misread, resulting, perhaps, in a misdiagnosis of the patient's condition. Further, with such awkward apparatus, a physician at times may choose to forego use of the manometer 120 due to inconvenience, thereby potentially failing to detect and diagnose a pathologically elevated or depressed pressure.

A need exists, therefore, for a more precise, accurate and/or convenient way to measure the pressure of a bodily fluid.

### BRIEF SUMMARY OF THE INVENTION

Various embodiments of the invention, therefore, provide enhanced devices for the measurement of a pressure of a bodily fluid, as well as methods of using such devices. In one set of embodiments, for example, a device for measuring a fluid may comprise an inlet port, which may be configured to be in fluid communication with a bodily fluid. Merely by way of example, in a particular set of embodiments, the inlet port may be in fluid communication with (and/or may comprise and/or be incorporated within) a needle designed to be inserted (e.g., subdermally) into a subject (such as a human and/or animal patient).

The inlet port may be in fluid communication with a pressure-responsive element, which may allow the fluid to exert a pressure on the pressure-responsive element. In some embodiments, a valve may provide fluid communication between the inlet port and the pressure-responsive device (and/or between a needle and the inlet port). An exemplary valve is a three-way stopcock.

In a particular set of embodiments, the pressure-responsive element may be designed to move and/or deform in response to the pressure exerted by the fluid. Merely by way of example, in some embodiments, the pressure-responsive element might comprise a diaphragm, which might be configured to deform (perhaps in a predictable fashion) when subjected to pressure from the fluid. In other embodiments, the pressure-responsive element might be configured to move (again, perhaps in predictable fashion) in response to the pressure exerted by the fluid. Operatively coupled to the pressure-responsive element may be a pressure sensor, which may be configured to sense the pressure exerted on the pressure-responsive element.

In accordance with various embodiments, any suitable type of pressure-responsive element and/or any suitable type of pressure sensor may be used. Merely by way of example, if the pressure-responsive element is a diaphragm that is configured to deform under pressure, the pressure sensor may comprise a strain gauge (or any other suitable device) and/or may be configured to measure the deformation of the pressure-responsive element. As another example, if the pressure-responsive element is configured to move in response to pressure, the pressure sensor may be a device configured to sense the motion of the pressure-responsive element. In particular embodiments, the pressure sensor might comprise (and/or be incorporated within) the pressure-responsive element.

The pressure sensor may also be in communication with a display device, which may be configured to provide a display of the pressure sensed by the pressure sensor. Any suitable display device may be used. In particular embodiments, for example, the display device is a digital display. In other embodiments, the display device may be an analog display (such as a dial, and/or the like). In further embodiments, the display device may be computer (and/or a computer display), that is in communication with the pressure sensor. The computer may be configured to display the measured pressure, create a record (such as a database record, and/or the like) of the measured pressure, analyze the measured pressure, and/or the like

The communication between the pressure sensor and the display device may be provided by any suitable communication medium. Merely by way of example, in some cases, the display may be incorporated within a body of a pressure-measurement device, such that integrated wiring provides the communication. In other embodiments, the display may be embodied by a separate enclosure, and/or a cable may provide communication between the pressure sensor and the display device. Thus, the display device may be in communication with the pressure sensor via wired communication. In yet other embodiments, such communications may be wireless, and/or the pressure-measurement device may comprise a wireless transmitter in communication with the pressure sensor. The display device, then, may feature a wireless receiver, which can be configured to receive wireless communication from the pressure sensor.

In some cases, a signal processor may be in communication with the pressure sensor and/or the display device. The signal processor may be configured to convert a signal from the pressure sensor to a signal that can be displayed by the display device. Merely by way of example (for instance, when the display device comprises a digital display), the signal processor may comprise an analog-to-digital converter.

In accordance with one set of embodiments, the device (and/or a portion thereof) may be configured to be disposed after a single use. In accordance with another set of embodiments, at least a portion of the device may be configured to be reusable. Merely by way of example, the display device might have a detachable connection that allows the display device to be removed from the rest of the pressure-measurement device, such that the display device can be reused, while the remainder of the pressure-measurement device may be discarded.

Another set of embodiments provides methods of measuring the pressure of a fluid, including without limitation a bodily fluid. An exemplary method may comprise providing a device for measuring the pressure of a fluid (such as, merely by way of example, any of the devices described above). The method may further comprise placing the inlet port of such a device in communication with a bodily fluid (for example, inserting a needle, which may be in communication with the inlet port and/or may comprise the inlet portion) into a spinal tap, and/or the like) and/or reading a display device to ascertain a pressure of the bodily fluid. In particular embodiments, the method comprises disposing of and/or reusing at least a portion of the device.

Yet another set of embodiments provides systems for measuring the pressure of a fluid. An exemplary system may comprise a pressure measurement device (including those described above, for example), and a computer system in communication (e.g., wired, wireless and/or the like) with the pressure measurement device. The computer system may comprise a processor and a computer readable medium having instructions executable by the processor to receive from the pressure sensor data about the pressure sensed by the pressure sensor and/or to perform an operation with respect to the data about the pressure sensed by the pressure sensor. Exemplary operations may include storing the data, analyzing the data, correlating the data with other patient data (including for instance, a patient's medical history, etc.), and/or displaying a pressure measurement based on the data (for instance on a computer display).

### BRIEF DESCRIPTION OF THE DRAWINGS

FIG. 1 illustrates a conventional device for measuring the pressure of a fluid in a patient.

FIG. 2 illustrates a device for measuring a fluid, in accordance with various embodiments of the invention.

FIGS. 3 and 4 illustrate exemplary pressure measurement devices in accordance with various embodiments of the invention.

FIG. 5 illustrates a schematic diagram of a pressure measurement device, in accordance with embodiments of the invention.

FIG. 6 illustrates a computer system comprising a pressure measurement device, in accordance with embodiments of the invention.

FIG. 7 illustrates a wireless communication system comprising a pressure measurement device, in accordance with embodiments of the invention.

FIG. 8 is a process flow diagram illustrating a method of measuring the pressure of a fluid, in accordance with embodiments of the invention.

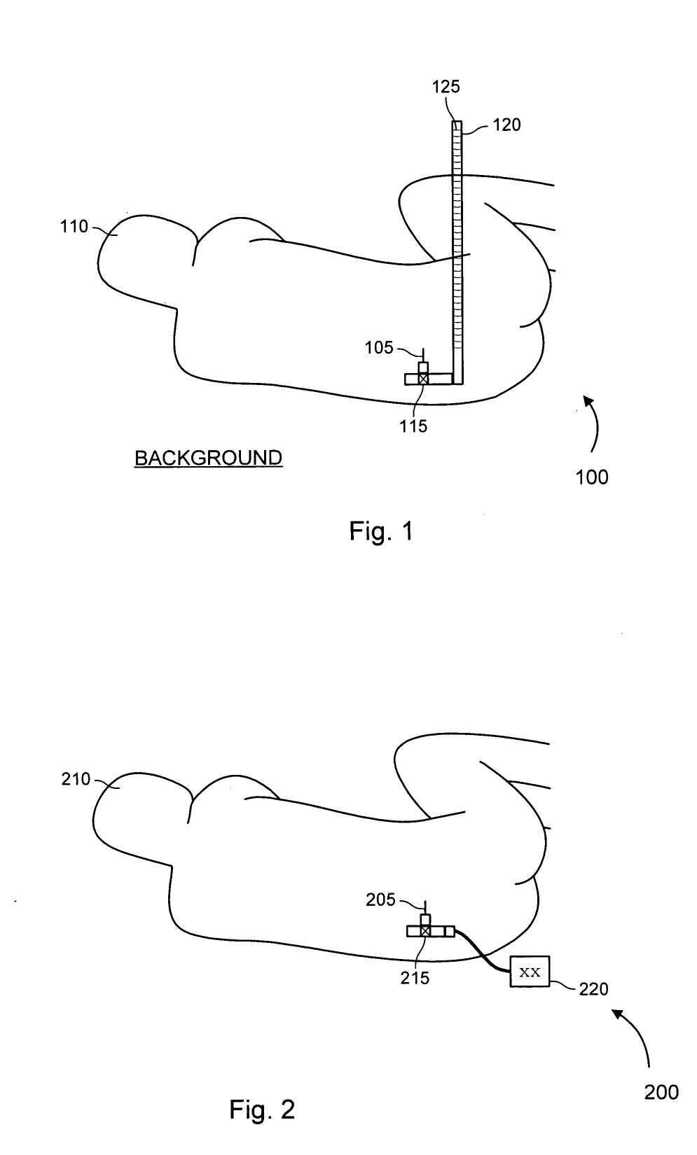

### DETAILED DESCRIPTION OF THE INVENTION

Various embodiments of the invention provide enhanced devices for the measurement of a bodily fluid, as well as methods of using and/or manufacturing such devices. In one set of embodiments, for example, a device for measuring a fluid may comprise an inlet port, which may be configured to be in fluid communication with a bodily fluid. Merely by way of example, in a particular set of embodiments, the inlet port may be in fluid communication with (and/or may comprise and/or be incorporated within) a needle designed to be inserted (e.g., subdermally) into a subject (such as a human and/or animal patient).

Merely by way of example, FIG. 2 illustrates a device 200 that can be used to measure the pressure of a fluid. The device 200, in certain embodiments, may be used to measure a bodily fluid, such as cerebrospinal fluid (“CSF”), blood, and/or any other suitable fluid. (It should be recognized, of course, that in accordance with alternative embodiments, devices similar to the device 200 and/or other devices described below may be used to measure any appropriate fluid, either in a biological context or otherwise.)

The device 200, therefore, may comprise a needle 205 and/or other means for accessing the fluid to be measured. In many cases, it may be desirable to measure the pressure of the fluid in situ, and the needle 205 therefore may be adapted to be inserted into a patent 210 (and/or any other subject). The needle 205 may be coupled with (and/or otherwise in fluid communication with) a main body 215, in which the pressure of a fluid (e.g., a fluid accessed with the needle 205) may be measured. The main body 215 may be coupled with and/or in communication (for example, as described in detail below) with a display 220, which can be used to display a pressure of the fluid.

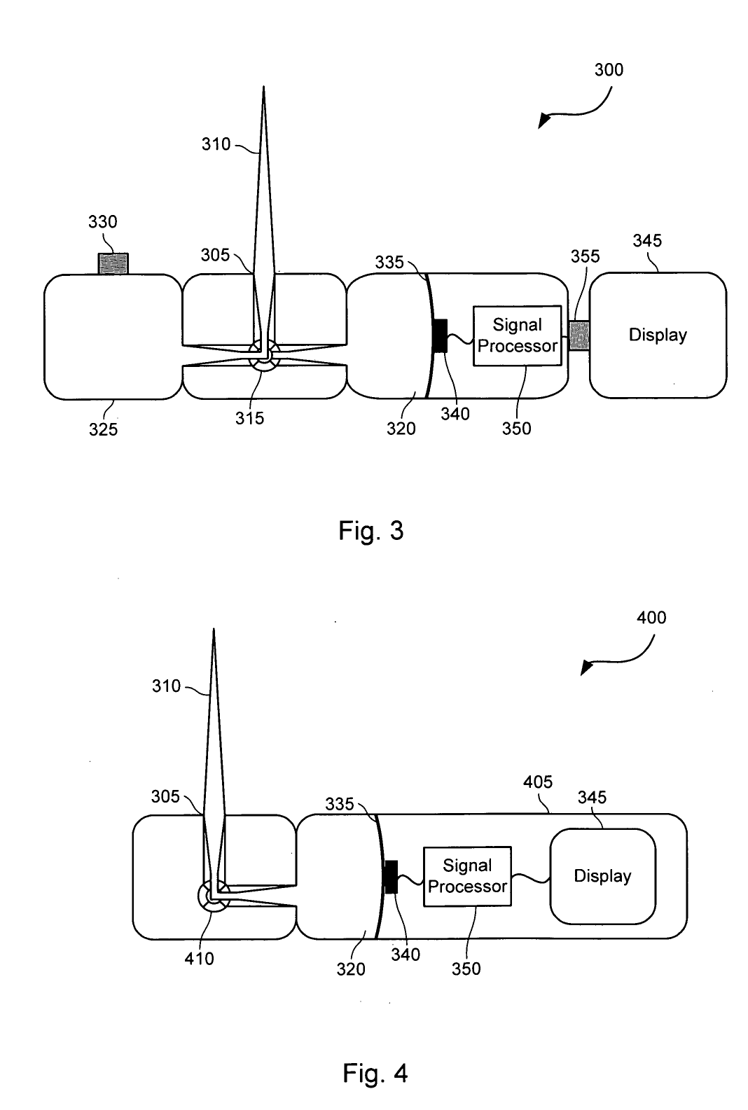

FIG. 3 is illustrates a pressure measurement device 300 in more detail. The device 300 may comprise an inlet port 305, which may be adapted (e.g., through any suitable fitting, of which many are commercially available) to be in fluid communication with a needle 310 (and/or any other component used to access a fluid, particularly in situ). In a set of embodiments, the inlet port 305 may actually comprise the needle 310, and/or the needle 310 and inlet port 305 may be integrally constructed. The inlet port 305 may be in fluid communication with (and/or may comprise) a valve 315, which may also be in fluid communication with a pressure measurement chamber 320, described in further detail below.

In accordance with various embodiments, any suitable valve 315 may be used (and in certain cases, the valve 315 may be omitted, providing direct fluid communication between the inlet port 305 and the pressure measurement chamber 320). Merely by way of example, in a set of embodiments illustrated by FIG. 3, a three-way stopcock is used. In alternative embodiments, other valves, including other stopcocks (e.g., four-way stopcocks) may be used. Depending on the embodiments, the device 300 may have an auxiliary chamber 325 and/or vent 330 in fluid communication with the valve 315. Either (or both) of the chamber 325 and/or vent 330 may be used to collect fluid samples and/or allow the escape of fluid from the device 300.

Merely by way of example, those skilled in the art will appreciate that a significant use of a spinal tap and/or lumbar puncture is to collect fluid for visual, chemical and/or biological analysis, and/or the like, and the vent 330 and/or auxiliary chamber 325 may be used for this purpose. Thus, the vent 330 and/or auxiliary chamber 325 may be in fluid communication with (and/or may be replaced with) a fluid collection apparatus. Alternatively and/or in addition, the pressure measurement chamber 320 may be configured to allow the collection of fluid in a similar manner and/or to be replaced with a fluid collection apparatus. Hence, embodiments of the invention may allow for the measurement of the pressure in a fluid without impeding the ability to collect fluid for analysis.

In certain embodiments, the pressure measurement chamber 320 may comprise and/or or enclose a pressure measurement apparatus, which can be used to measure the pressure of a fluid in the pressure measurement chamber 320. The pressure measurement apparatus may comprise a pressure-responsive element (which may be configured to deform, move, and/or the like in response to a pressure applied by the fluid), along with a pressure sensor, which can sense the pressure in the fluid, perhaps based on the behavior of the pressure-responsive element. In particular embodiments, the pressure-responsive element and the pressure sensor may be integrated in a single apparatus.

A variety of different types of pressure measurement apparatus may be used. Merely by way of example, as depicted in FIG. 3, the pressure-responsive element may comprise a diaphragm 335 (and/or any other deformable element). The diaphragm 335 may be composed of any suitable material, including a variety of plastics, rubbers, metals and/or the like. In a set of embodiments, the diaphragm 335 comprises a material and/or construction that provides a predictable deformation curve based on the amount of pressure applied to the diaphragm. In another set of embodiments, the diaphragm 335 may comprise a material that is resistant to reaction with the types of fluids being measured. The pressure sensor 340, then, may be a strain gauge and/or any other type of device that can function to measure the deformation of the diaphragm 335. A variety of suitable pressure sensors are available. Merely by way of example, models SM5852, SM5420, SM5430, SM5470, SM5108, SM5112, SM5103, all commercially available from Silicon Microstructures, Inc. of Milpitas, Calif., may be suitable for use with embodiments of the invention. Other pressure-responsive elements and/or pressure sensors (including without limitation those described below) may be used as well.

The pressure measurement chamber 320 (and/or more specifically, the pressure sensor 340) may be coupled (e.g., electronically, mechanically, and/or the like) with a display device 345, which may be configured to display a pressure sensed by the pressure sensor 345. The display device 345 may be a digital display, an analog display, a pseudo-analog display, a gauge, a dial, and/or the like. Merely by way of example, a digital display might display a digital reading of the pressure of the fluid. In particular embodiments, for example, the display device 345 may comprise a LCD and/or LED readout that provides a pressure indication in digital fashion. Merely by way of example, the model DMS30-LCD-1-5B display, commercially available from Datel, Inc. of Mansfield, Mass., and/or the model VI-321 display, commercially available from Varitronix International Limited of Hong Kong, may be used in accordance with some embodiments. Many other suitable alternatives are available as well. Alternatively and/or in addition, an analog display and/or a pseudo-analog display (i.e., a digital display that is configured to appear as an analog display) might comprise a meter, dial, bar graph and/or the like.

In accordance with some embodiments, the display might be configured to provide a visual indication of whether the pressure of the fluid falls within a safe zone. Merely by way of example, some embodiments feature a digital display configured to display pressures considered safe and/or healthy using a particular color (e.g., green), pressures bordering on unsafe using another color (e.g., yellow) and/or pressures considered definitely unsafe and/or pathological using a third color (e.g., red). (Of course, those skilled in the art will appreciate, based on the disclosure herein, that any number of gradations and/or colors may be implemented in accordance with various embodiments). Similarly, other embodiments feature an analog and/or pseudo-analog display configured to provide a visual indication of whether the pressure of the fluid is in a safe range. Merely by way of example, a gauge, dial, bar graph and/or the like might be configured to display zones (e.g., green, yellow, red, as described above), corresponding to various pressures, such that an operator of the device 300 can easily determine at a glance whether the pressure measured falls into a safe zone, unsafe zone, and/or the like. In this way, embodiments of the invention can allow an operator to determine quickly, and without necessarily having to determine a precise pressure of the fluid, whether the pressure of the fluid indicates a pathology. In particular embodiments, the device 300 might also (or instead) be configured to provide an audio indication (e.g., using a speaker and/or the like) of whether the pressure falls within a safe zone, for instance by sounding an alarm if the pressure does not fall within a range considered safe and/or by playing different tones based on the pressure measured (e.g., a first tone if the pressure is within a range considered safe and/or healthy, and a second tone if the pressure is not within a range considered safe and/or healthy).

In some cases, the device 300 may be pre-configured to indicate certain zones and/or ranges in a certain manner (e.g., with certain colors, audio tones, etc.). In other cases, the device 300 may be configurable by an operator (and/or another) to allow customization of this feature. Merely by way of example, an operator could define a particular range of pressures as safe, with other ranges being defined as borderline and/or unsafe, and the device then would operate accordingly.

In particular embodiments, for example as described in further detail below, the display device 345 may comprise, and/or be incorporated in, a computer and/or computer display. Further, in various embodiments, the pressure sensor 340 may be in communication with the display device 345 via any of a variety of communication means, including without limitation wired communication, wireless communication, and/or the like.

Depending on the embodiment, it may be appropriate to condition and/or otherwise process data produced by the pressure sensor 340 prior to reception by the display device 345. Accordingly, particular embodiments may feature a processor, microprocessor and/or signal processor 350 configured to to format, convert and/or otherwise process a signal produced by the pressure sensor 340. Merely by way of example, as noted above, certain embodiments feature a digital display. It is common for pressure sensors to produce analog data; a signal processor 350, therefore, may comprise an analog-to-digital converter (“ADC”), which can allow a digital display to receive and/or display signals produced by an analog device. The signal processor 350, therefore, may be in communication with the pressure sensor 340 and/or the display device 345. Similarly, an ADC may be used to format the data produced by the pressure sensor 340 for reception and/or storage by a computer. Other types of signal processors may be used as well. Merely by way of example, a microprocessor with a display driver, such as, for instance, the MSP430C113, commercially available from Texas Instruments, Inc. of Dallas, Tex. may be used. Other embodiments may utilize a microprocessor without an integrated display driver, such as the AT89C4051, commercially available from Atmel Corp. of San Jose, Calif. Of course, those skilled in the art will appreciate, based on the disclosure here, that a variety of processors and/or microprocessors may be used in accordance with embodiments of the invention.

Those skilled in the art will appreciate that, in many circumstances, it may be desirable for a pressure measurement device to be disposable. For example, for the sake of convenience and/or hygiene, it may be suitable to provide single-use pressure measurement devices. Accordingly, certain embodiments of the invention comprise relatively inexpensive components, allowing a pressure-measurement device to be produced and/or procured relatively inexpensively. In other embodiments, it may be more cost-effective to allow some (or all) of the components to be reused. Merely by way of example, the display device and/or processor may be relatively expensive components, such that it might be suitable to reuse the display device and/or processor while discarding the remainder of the pressure measurement device. Accordingly, one or more components (such as the display device 345 and/or others) may be provided with detachable connectors (including connectors known in the art), allowing those components to be removed from the pressure measurement device 300 and/or reattached to another such device. The remainder of the pressure measurement device 300 then may be disposable.

Merely by way of example, the pressure measurement device 300 of FIG. 3 provides a fitting 355 to allow the display 345 to be removably coupled with the remainder of the pressure measurement device 300. (Alternatively, the fitting 355 may provide for the detachment of the processor 350 and the display device 345, which may be integrated together within a single housing, to be detached from the remainder of the pressure measurement device 300.) Any suitable fitting may be used. Merely by way of example, threaded fittings, bayonet fittings, coaxial fittings, male/female connectors and/or the like may be provided. In particular embodiments, the fitting 350 may be configured to provide electrical communication and/or mechanical coupling between the display 345 (or the processor 350) and the remainder of the pressure measurement device 300. Several types of commercially-available fittings can provide this functionality, including without limitation, threaded coaxial fittings and the like. Alternatively, separate mechanical (such as a threaded fitting, a snap-on fitting, and/or the like) and electrical (such as a wiring harness, and/or the like) connectors may be implemented.

In another set of embodiments, an integrated housing may incorporate both a pressure measurement chamber (along with a pressure measurement apparatus) and a display device, as well, perhaps, as a needle (and/or a connector therefore). In some cases, therefore, a pressure measurement device may be embodied by a unitary construction, such as a molded housing, and/or the like In particular cases, the entire device may be designed to be disposable and/or reusable.

FIG. 4 illustrates an example of a pressure measurement device 400 incorporating a unitary housing, in accordance with some such embodiments. The device 400 illustrated by FIG. 4 is similar to the device 300 described above and illustrated by FIG. 3, and may be operated in a similar manner. The device 400 of FIG. 4, however, features a display device 345 integrated with the pressure measurement chamber 320 and other components within a unitary housing 405. Such embodiments, for example, may be configured to be disposed of (or reused) in their entirety.

For purposes of illustration, the valve 410 of the device 400 depicted in FIG. 4 is equipped with a two-way stopcock, and the device 400 does not feature an auxiliary chamber or vent. It should be noted, however, that the features described above with respect to FIG. 3 could be implemented in a unitary device similar to that of FIG. 4. (For that matter, as noted above, any suitable valve configuration may be used.)

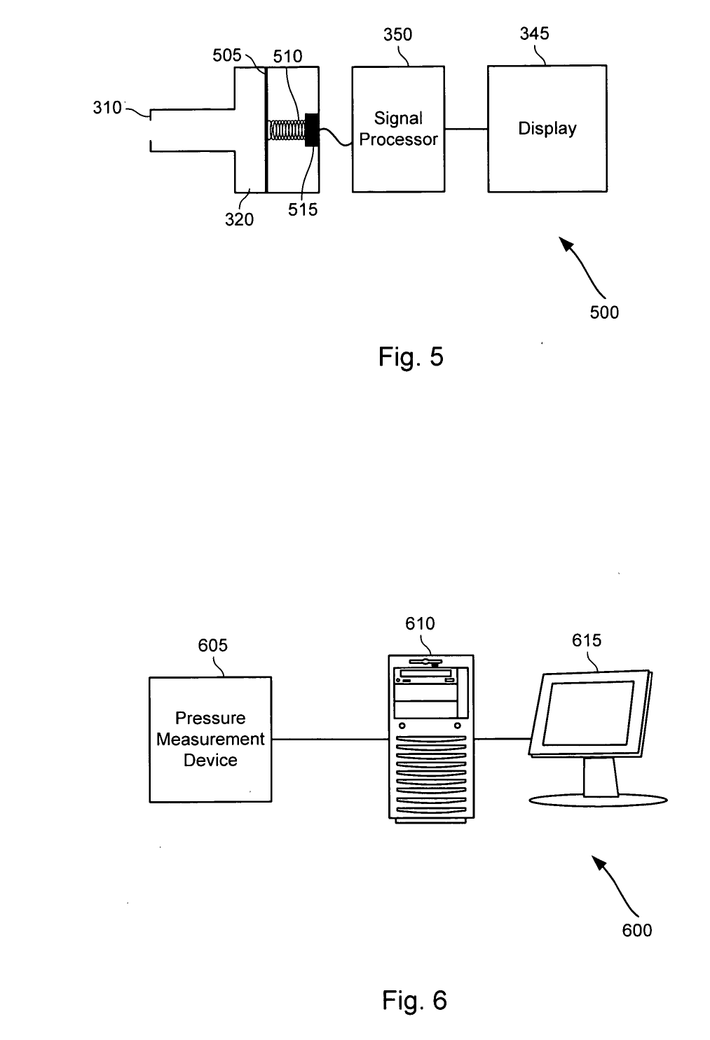

FIG. 5 illustrates a schematic diagram of an alternative embodiment of a pressure measurement device 500. The device 500 may have an inlet port 310, pressure measurement chamber 320, display device 345 and/or signal processor 350 similar to those described above. The device 500, however, features a rigid pressure responsive element 505, which may be configured to be displaced in response to a pressure exerted by a fluid in the pressure measurement chamber 320. Merely by way of example, the pressure responsive element 505 may be a relatively rigid plate that is disposed within the pressure measurement chamber 320 in such a way to allow lateral movement in response to lateral forces. An elastic member 510 (such as a spring, elastomer, and/or the like) may be coupled with (and/or disposed against) the pressure responsive element 505, to provide resistance against movement of the pressure responsive element 505 in response to a fluid pressure in the pressure measurement chamber 320. The magnitude of the displacement of the pressure responsive element 505, then, may be measured to determine the pressure of the fluid. The magnitude of the displacement and/or the pressure of the fluid may be measured in several ways. Merely by way of example, the force exerted by the elastic member 510 on the pressure sensor 515. may be measured, and/or the pressure of the fluid could be calculated from this measurement. Alternatively, the pressure sensor 510 could comprise a position sensor that could be used to determine the position of the pressure responsive element 505. Based on the displacement of the pressure-responsive element 505 and/or the spring constant (or some other value) of the elastic member 510, the pressure of the fluid may be calculated. Those skilled in the art will appreciate, based on the disclosure herein, that a variety of methods may be used to calculate the pressure of the fluid in such embodiments.

As noted above, in some instances, a pressure measurement device may be in communication with a computer, which may be used to track, record, store and/or display pressures measured by such a device. FIG. 6 illustrates an exemplary system 600 that may be used for such purposes. The system 600 may comprise a pressure measurement device 605 (which may be similar to any of the devices described above), which may be in communication with a computer 610 having a display 615. In some embodiments, the computer 610 may be in communication with a signal processor (such as those described above, for example). In other embodiments, the computer 610 may be in communication with a pressure and/or location sensor. In such embodiments, the computer may comprise an ADC for converting analog data received from the sensor(s). The device 605 (and/or components thereof) may feature any standard connection for communicating with a computer, including without limitation wired connections (such as a serial connection, USB connection and/or any other standard or proprietary connection), as well as wireless connections, including for example those described below.

The computer 610, which may be a standard personal computer, workstation, laptop, and/or the like, may also be configured with software designed to receive, process and/or display data produced by the pressure measurement device 605 (and/or any suitable components thereof). Merely by way of example, the computer 610 may comprise an application program configured to convert raw data received from the pressure measurement device 605 into calibrated pressure measurements Alternatively and/or in addition, the computer 605 may comprise an application program configured to store pressure measurements (which could allow, for example, historical analysis of pressure measurements over time), to analyze pressure measurements (which could allow, for example, the computer to assist in the diagnosis of conditions, and/or the like). In particular embodiments, such an application program may be configured to correlate pressure measurements with other data (such as a medical history of a patient, and/or the like). The computer 610 also may be configured to display pressure measurements on the display 615. Thus, the display 615, in conjunction with the computer 610, can serve as the display device for the pressure measurement device 605.

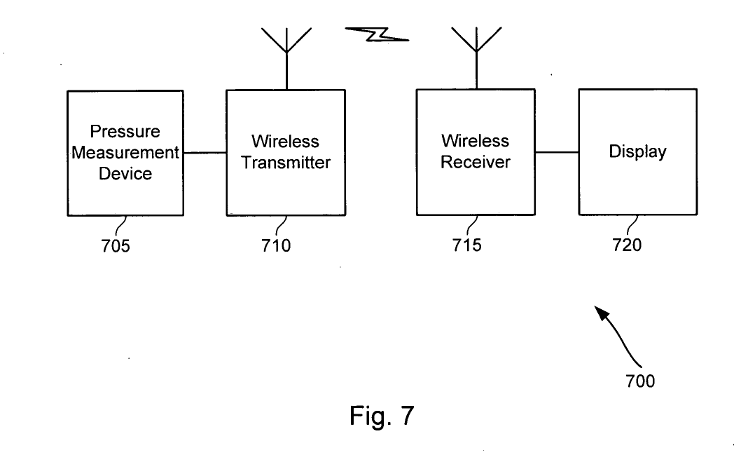

A particular set of embodiments provides for wireless communication between a pressure measurement device and a display device (and/or a computer, which, as noted above can also serve as a display device). FIG. 7 illustrates an exemplary system 700 that can be used to provide such wireless communication. The system 700 comprises a pressure measurement device 700, which may be similar to any such device described above. The pressure measurement device 700 (and/or a component thereof, such as a processor, sensor, and/or the like) is in communication with a wireless transmitter 710, which is configured to transmit a wireless signal for reception by a wireless receiver 715. The wireless receiver, then, may be in communication with a display device 720, a computer, and/or the like Thus, the system 700 may function similarly to pressure measurement devices and/or systems described above, but the display device 720, computer, and/or the like need not be in physical communication with the pressure measurement device.

Any suitable variety of wireless communication may be supported by the wireless transmitter 710 and/or receiver 715. Merely by way of example, in a set of embodiments, the wireless transmitter 710 is configured to transmit data via a Bluetooth™ interface for reception by the wireless receiver 715. The wireless receiver 715, then, may be any device that is capable of receiving Bluetooth™ communications. In other embodiments, the wireless transmitter 710 and/or receiver may be configured to use any of the IEEE 802.11 suite of protocols, any suitable infrared protocol, and/or the like. Based on the disclosure herein, one skilled in the art will appreciate that many wireless protocols could be used in accordance with various embodiments of the invention.

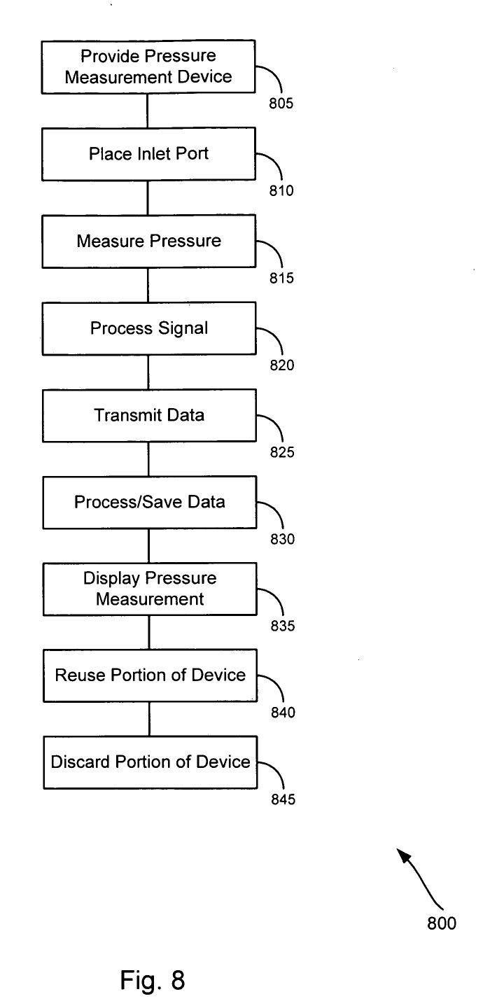

Another set of embodiments provides methods for using a pressure measurement device. FIG. 8 illustrates an exemplary method 800. The method 800 comprises providing a pressure measurement device (block 805), which may be, inter alia, any of the devices described above. The inlet port of the pressure measurement device is placed in communication with a fluid (block 810). In some embodiments, placing the inlet port in communication with a fluid may comprise inserting a needle into a patient (e.g., into the subarachnoid space). At this point, samples of the fluid may be taken if appropriate.

The pressure of the fluid then may be measured (block 815), e.g., by allowing the fluid to flow into a pressure measurement chamber (perhaps through the use of a valve) and sensing (with a pressure sensor, location sensor and/or the like, as described above) the pressure exerted by the fluid on a pressure-responsive element. In accordance with some embodiments, the signal from a sensor may need to be processed (block 820) (e.g., to convert an analog signal to a digital signal, to perform a data transformation, and/or the like).

Data about the measured pressure then may be transmitted (block 825). In accordance with some embodiments, the data may be transmitted for reception by a display device. As noted above, other embodiments of the invention feature a computer. Accordingly, data also may be transmitted for reception by the computer. As noted above, data transmission may be accomplished via any suitable procedure, using wired and/or wireless media. In some cases (such as, for instance, when data is transmitted to a computer), the data may be processed and/or saved, perhaps as described above. The pressure measurement then may be displayed (block 835), e.g., by a display device, a computer display, and/or the like.

As noted above, in accordance with particular embodiments, some or all portions of a pressure measurement device may be reusable and/or disposable. The method thus may comprise reusing at least a portion of the device (block 840), such as a display, and/or discarding at least a portion of the device (block 845), such as a needle, pressure measurement chamber and/or the like.

In conclusion, embodiments of the present invention provide pressure measurement devices and methods for their use. While detailed descriptions of one or more embodiments of the invention have been given above, various alternatives, modifications, and equivalents will be apparent to those skilled in the art without varying from the spirit of the invention. Moreover, except where clearly inappropriate or otherwise expressly noted, it should be assumed that the features, devices and/or components of different embodiments can be substituted and/or combined. Thus, the above description should not be taken as limiting the scope of the invention, which is defined by the appended claims.

What is claimed is:

1. A device for measuring a pressure of a bodily fluid, the device comprising:
an inlet port configured to be in fluid communication with a bodily fluid;
a pressure-responsive element in fluid communication with the inlet port, such that the bodily fluid exerts a pressure on the pressure-responsive element;
a pressure sensor operatively coupled with the pressure-responsive element and configured to sense the pressure exerted on the pressure-responsive element; and
a display device in communication with the pressure sensor and configured to display the pressure sensed by the pressure sensor.

2. A device for measuring a pressure of a bodily fluid as recited in claim 1, wherein the bodily fluid is cerebrospinal fluid.

3. A device for measuring a pressure of a bodily fluid as recited in claim 1, wherein the inlet port is configured to be in fluid communication with a spinal tap needle.

4. A device for measuring a pressure of a bodily fluid as recited in claim 1, wherein the inlet port comprises a spinal tap needle.

5. A device for measuring a pressure of a bodily fluid as recited in claim 1, wherein the pressure-responsive element comprises a diaphragm.

6. A device for measuring a pressure of a bodily fluid as recited in claim 5, wherein the diaphragm is configured to deform in response to the pressure exerted by the bodily fluid.

7. A device for measuring a pressure of a bodily fluid as recited in claim 6, wherein the pressure sensor is configured to measure a deformation of the diaphragm.

8. A device for measuring a pressure of a bodily fluid as recited in claim 7, wherein the pressure sensor comprises a strain gauge.

9. A device for measuring a pressure of a bodily fluid as recited in claim 5, wherein the diaphragm is configured to be displaced in response to the pressure exerted by the bodily fluid.

10. A device for measuring a pressure of a bodily fluid as recited in claim 9, wherein the pressure sensor is configured to measure a displacement of the diaphragm.

11. A device for measuring a pressure of a bodily fluid as recited in claim 1, wherein the display device comprises a digital display.

12. A device for measuring a pressure of a bodily fluid as recited in claim 1, wherein the display device comprises an analog display.

13. A device for measuring a pressure of a bodily fluid as recited in claim 1, wherein the display device comprises a pseudo-analog display.

14. A device for measuring a pressure of a bodily fluid as recited in claim 1, wherein the display device is configured to provide an indication whether the pressure of the bodily fluid falls within a safe zone.

15. A device for measuring a pressure of a bodily fluid as recited in claim 14, wherein the indication is a visual indication.

16. A device for measuring a pressure of a bodily fluid as recited in claim 14, wherein the indication is an audio indication.

17. A device for measuring a pressure of a bodily fluid as recited in claim 1, wherein the display device is associated with a computer, such that computer displays the pressure sensed by the pressure sensor.

18. A device for measuring a pressure of a bodily fluid as recited in claim 17, wherein the computer is configured to record the pressure sensed by the pressure sensor.

19. A device for measuring a pressure of a bodily fluid as recited in claim 1, wherein the display device is in communication with the pressure sensor via wireless communication.

20. A device for measuring a pressure of a bodily fluid as recited in claim 1, wherein the display device is in communication with the pressure sensor via wired communication.

21. A device for measuring a pressure of a bodily fluid as recited in claim 11, the device further comprising:
a processor in communication with the pressure sensor and the display device, the processor being configured to convert a first signal from the pressure sensor to a second signal that can be displayed by the display device.

22. A device for measuring a pressure of a bodily fluid as recited in claim 21, wherein the signal processor comprises an analog-to-digital converter.

23. A device for measuring a pressure of a bodily fluid as recited in claim 1, further comprising a valve in fluid communication with the inlet port and the diaphragm, the valve being configured to selectively provide fluid communication between the inlet port and the diaphragm.

24. A device for measuring a pressure of a bodily fluid as recited in claim 23, wherein the valve comprises a three-way stopcock.

25. A device for measuring a pressure of a bodily fluid as recited in claim 1, wherein at least a portion of the device is configured to disposed after a single use.

26. A device for measuring a pressure of a bodily fluid as recited in claim 1, wherein the entire device is configured to be disposed after a single use.

27. A device for measuring a pressure of a bodily fluid as recited in claim 1, wherein at least a portion of the device is configured to be reusable.

28. A method of measuring a pressure of a bodily fluid, the method comprising:
providing a device for measuring a pressure of a bodily fluid, the device comprising:
an inlet port configured to be in fluid communication with a bodily fluid;
a pressure-responsive element in fluid communication with the inlet port, such that the bodily fluid exerts a pressure on the pressure-responsive element;
a pressure sensor operatively coupled with the pressure-responsive element and configured to sense the pressure on the pressure-responsive element; and
a display device in communication with the pressure sensor and configured to display the pressure sensed by the pressure sensor;
placing the inlet port of the device in communication with a bodily fluid; and
reading the display device to ascertain a pressure of the bodily fluid.

29. A method of measuring a pressure of a bodily fluid as recited in claim 28, the method further comprising:
disposing of at least a portion of the device.

30. A method of measuring a pressure of a bodily fluid as recited in claim 28, the method further comprising:
reusing at least a portion of the device.

31. A method of measuring a pressure of a bodily fluid as recited in claim 28, wherein placing the inlet port of the device in communication with a bodily fluid comprises inserting a needle into a spinal tap.

32. A system for measuring the pressure of a bodily fluid, the system comprising:
a pressure measurement device, comprising:
an inlet port configured to be in fluid communication with a bodily fluid;
a pressure-responsive element in fluid communication with the inlet port, such that the bodily fluid exerts a pressure on the pressure-responsive element; and
a pressure sensor operatively coupled with the pressure-responsive element and configured to sense the pressure exerted on the pressure-responsive element; and
a computer system in communication with the pressure measurement device, the computer system comprising a processor and a computer readable medium having instructions executable by the computer to:
receive from the pressure sensor data about the pressure sensed by the pressure sensor; and
perform an operation with respect to the data about the pressure sensed by the pressure sensor.

33. A system for measuring the pressure of a fluid as recited in claim 32, wherein the operation is selected from the group consisting of:
storing the data;
analyzing the data;
correlating the data with other patient data; and
displaying on a computer display a pressure measurement based on the data.

34. A system for measuring the pressure of a fluid as recited in claim 32, wherein the computer system is in wireless communication with the pressure measurement device.
